(19) United States
(12) Patent Application Publication
(10) Pub. No.: US 2017/0347858 A1
(43) Pub. Date: Dec. 7, 2017

(54) CONTROLLING APPARATUS FOR BENDING THE TUBE OF AN ENDOSCOPE

(71) Applicant: CHIYI TECHNOLOGY CO., LTD., NEW TAIPEI CITY (TW)

(72) Inventor: YEN TSUNG LIN, NEW TAIPEI CITY (TW)

(21) Appl. No.: 15/171,132

(22) Filed: Jun. 2, 2016

(51) Int. Cl. A61B 1/005

(52) U.S. Cl. CPC A61B 1/0052

(57) ABSTRACT

The present invention relates to a controlling apparatus for bending the tube of an endoscope, in which the tube of the endoscope is consisted of different rigid and soft parts. The tail portion of the tube is provided at least with a soft foldable ring and other rigid portion becomes a support base. A slot is formed in the tube for receiving a controlling slice, which has an elastic end being bent in tenseness and the other end extending into a handle to engage with a teeth piece. A gear set provided within the handle engages with the teeth piece. By turning the gear set, it can move the teeth piece as well as the controlling slice toward the tail portion of the tube to control the tail portion being bent under simple operation. When the controlling slice moves towards the handle and is apart from the soft foldable ring of the tube, the tail portion will be recovered flatly because of being lack of elastic force. It therefore obtains improvement and utility that the present invention is capable of bending the tail portion of the tube easily.

### BACKGROUND OF THE INVENTION

The endoscope normally utilized on medical surgery procedures that is harmless to people, includes a long tube for inserting into human's body. The tail portion of the tube is provided with a camera module and a lighting source therein to catch image data of organs in human's body. In order to obtain more images, the conventional structure is provided with connecting wires in the tube to control the tail portion being able of bending. As shown in FIGS. 1 and 2, the controlling apparatus of known endoscope is provided with several units (1) having a cylinder body (11) and two joint portions (12). Each unit (1) has its two joint portions (12) engaged with the body (11) of another unit by respective micro rivets (13) to become a bendable tail portion. In this way, it is obviously inconvenience in connecting procedures of such small units and the connection of controlling wires is very difficult. Moreover, the traditional operating procedure is not easy for medical operators. Other conventional endoscopes, such as the hysteroscopy or laparoscopic, usually have a structure having its tail portion of the tube being bent to become a fixed angle. Such apparatus is not convenient to be inserted into the patient's body and will course uncomfortable feeling since the medical operator must turn the tube frequently in order to find different images of related organs of patients. Hence, the known designs are not utilized and should be improved.

### SUMMARY OF THE INVENTION

The present invention is aimed to design an invented controlling apparatus for bending the tube of an endoscope which can overcome the drawbacks mentioned in prior art, in which the tube of the endoscope is consisted of different rigid and soft parts. The tail portion of the tube is provided at least with a soft foldable ring and other rigid portion becomes a support base. A slot is formed in the tube for receiving a controlling slice, which has an elastic end being bent in tenseness and the other end extending into a handle to engage with a teeth piece. A gear set provided within the handle engages with the teeth piece. By turning the gear set, it can move the teeth piece as well as the controlling slice toward the tail portion of the tube. When the controlling slice moves to the position of the soft foldable ring, the tail portion will be bent by elastic force of the elastic end that reaches the purpose to control the tail portion of the tube being bent under simple operation. When the controlling slice moves towards the handle and is apart from the soft foldable ring of the tube, the tail portion will be recovered flatly because of being lack of elastic force. It therefore obtains improvement and utility that the present invention is capable of bending the tail portion of the tube easily. Accordingly, the tube of the present invention can be inserted into human's organs at flat configuration and then the tail portion can be bent easily to detect and obtain the situation in the organs of a patient who can suffer less uncomfortable feeling. Now, accompanying with drawings, the character and structure of the present invention will be disclosed as following.

### BRIEF DESCRIPTION OF THE DRAWINGS

FIG. 1 is a perspective view showing the controlling apparatus of a conventional endoscope.

FIG. 2 is the controlling apparatus being bent of FIG. 1.

FIG. 3 is a cross-sectional plan view of parts of an endoscope according to the present invention.

FIG. 4 is a cross-sectional plan view showing the tail portion being bent according to the present invention.

FIG. 5 is another embodiment of FIG. 4

FIGS. 6 and 7 show different embodiments of FIG. 4

FIG. 8 shows a modified embodiment with two controlling apparatuses according to the invention.

FIG. 9 is a cross-sectional plan view showing the tail portion being bent towards an opposed direction.

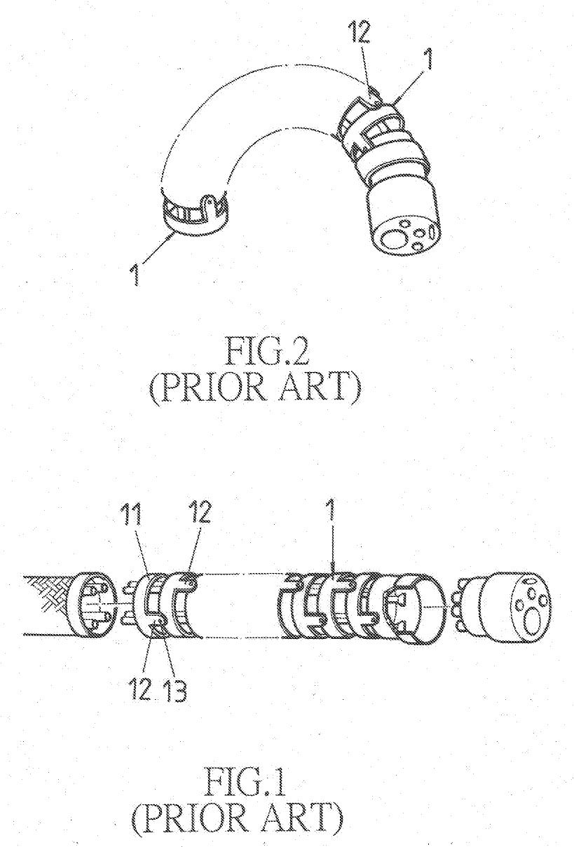

### DETAILED DESCRIPTION OF THE PREFERRED EMBODIMENT

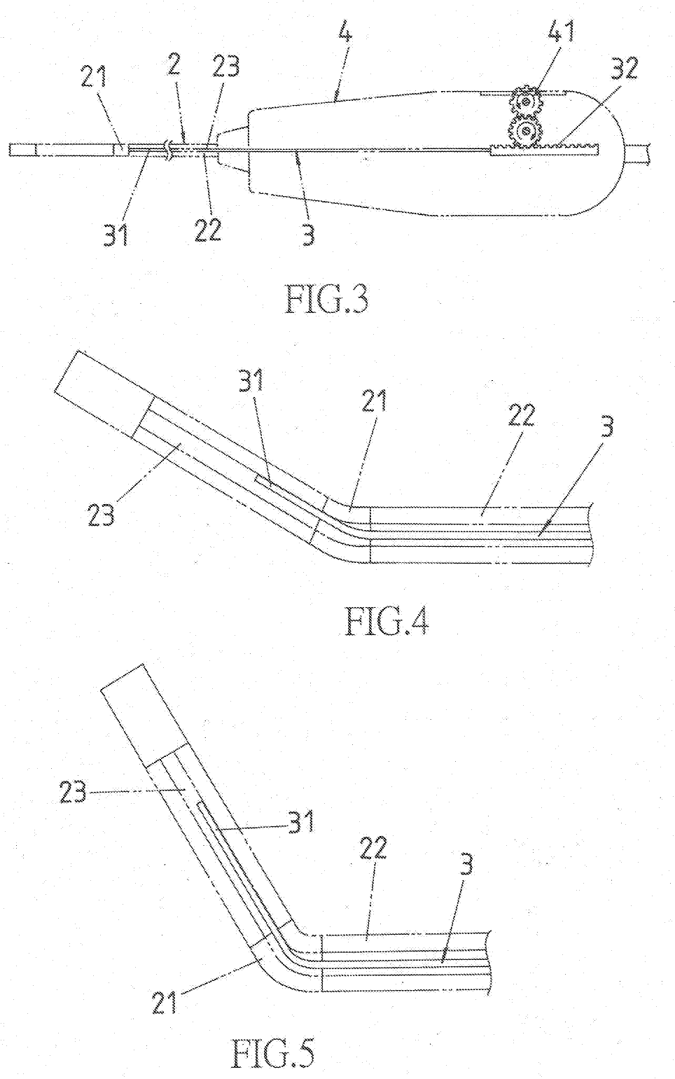

Please refer to FIGS. 3 and 4. The present invention relates to a controlling apparatus for bending the tube of an endoscope. The tube (2) of the endoscope is consisted of different rigid and soft parts, wherein the tail portion is provided at least with a soft foldable ring (21) and other rigid portion becomes a support base (22). A slot (23) is formed in the tube (2) for receiving a controlling slice (3) which is made of steel being high temperature dealing and then has an elastic end (31) being bent in tenseness. The other end of the controlling slice (3) is extending into a handle (4) to engage with a teeth piece (32) therein. A gear set (41) provided within the handle (4) engages with the teeth piece (32). By turning the gear set (41), it can move the teeth piece (32) as well as the controlling slice (3) toward the tail portion of the tube (2). When the controlling slice (3) moves to the position of the soft foldable ring (21), the tail portion will then be bent by elastic force of the elastic end (31), as shown is FIG. 4, that reaches the purpose to control the tail portion of the tube (2) being bent under simple operation. When the controlling slice (3) moves towards the handle (4) and is apart from the soft foldable ring (21) of the tube (2), the tail portion will immediately be recovered flatly, as in FIG. 3, because of being lack of elastic force. In order to bend the tail portion to a larger angle as in FIG. 5, the controlling slice (3) can move more toward to the end of the tube (2) that provides more elastic force to bend the tail portion having a large angle. By use of engagement between the gear set (41) with the teeth piece (32), the tail portion of the tube (2) can be bent to different angles and fixed at the position stably that will be useful for medical procedures.

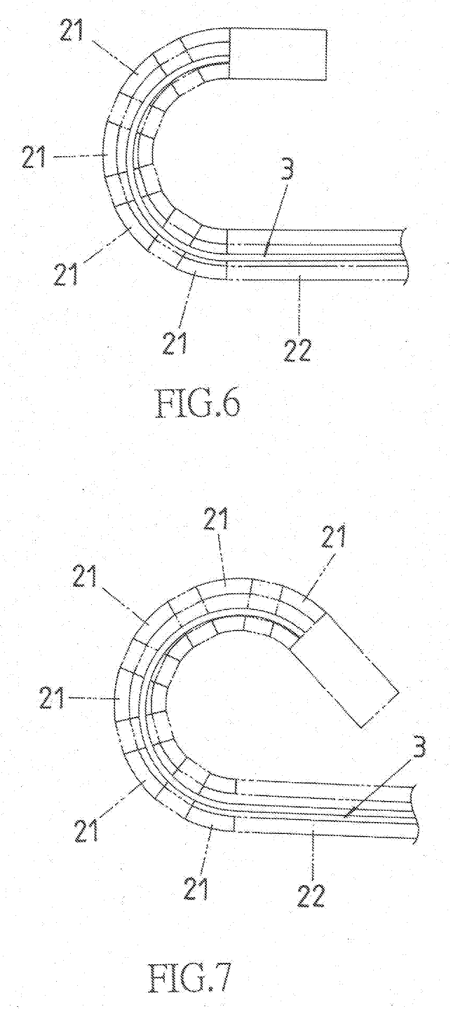

FIGS. 6 and 7 show another embodiment according to the present invention, wherein the tail portion of the tube (2) is provided with several soft foldable rings (21) in intervals. When the elastic end (31) of the controlling slice (3) reaches the soft foldable rings (31) serially, the tail portion of the tube (2) is then bent continuously and can be curved even to 270 degrees. It provides a better effect to obtain inner images of human organs.

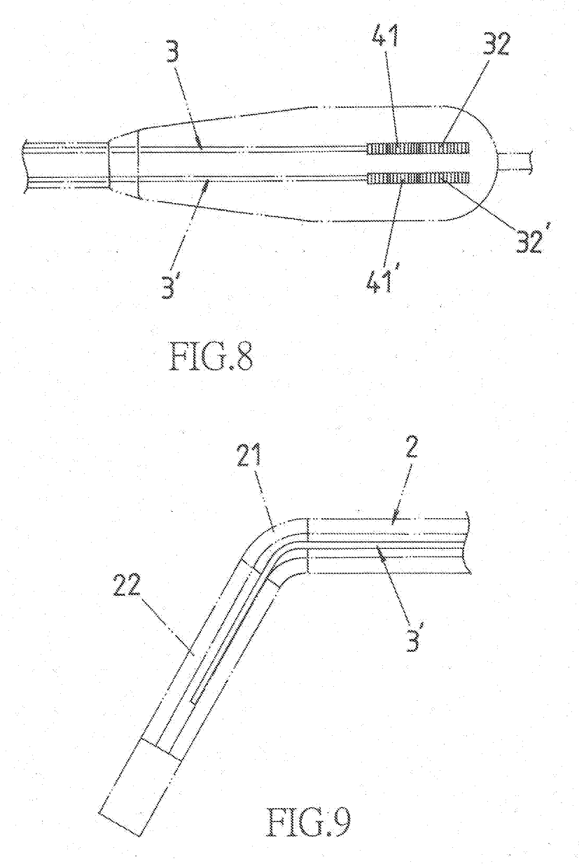

Please refer to FIGS. 8 and 9. The present invention can be provided with two controlling apparatus for controlling two bending directions of the tube. That is the endoscope has two controlling slices (3)(3′) received in the slot (23) engaging with two teeth pieces (32)(32′) and two gear sets (41)(41′) respectively. To operate the second controlling slice (3′), the teeth piece (32′), and the gear set (41′), the tail portion of the tube (2) can be bent to a reverse direction. Hence, this embodiment of the present invention can provide an invented endoscope having two dimension for taking images to facilitate the operation or detection during surgery.

Above all, the present obtains utility in no doubt. It can be used easily for patient in medical procedures. The tail portion of the tube of the endoscope can be controlled to bend easily and fixed at a desired position stably while it can obtain more detailed images of patient inner organs efficiently. Thus, it is to be understood that the present is allowable.

What is claimed is:

1. A controlling apparatus for bending the tube of an endoscope, wherein the tube of the endoscope having its tail portion provided at least with a soft foldable ring and other rigid portion becomes a support base, a slot formed in the tube for receiving a controlling slice, which has an elastic end being bent in tenseness and the other end extending into a handle to engage with a teeth piece,
a gear set provided within the handle to engage with the teeth piece, and
by turning the gear set to move the teeth piece as well as the controlling slice toward the tail portion of the tube to control the tail portion being bent, and moving the controlling slice towards the handle and being apart from the soft foldable ring of the tube, the tail portion then recovered flatly.

2. The controlling apparatus for bending the tube of an endoscope as claimed in claim 1, wherein the tail portion of the tube is bent during turning the gear set to move the teeth piece and the controlling slice, and the tail portion is capable of being fixed at a stable angle.

3. The controlling apparatus for bending the tube of an endoscope as claimed in claim 1, wherein the tail portion is provided with several soft foldable rings in intervals.

4. The controlling apparatus for bending the tube of an endoscope as claimed in claim 1, wherein the endoscope has two controlling slices received in the slot to engage with two teeth pieces and two gear sets respectively, while the second controlling slice, the teeth piece, and the gear set are provided for bending the tail portion of the tube to a reverse direction.
